(19) United States
(12) Patent Application Publication
(10) Pub. No.: US 2016/0108357 A1
(43) Pub. Date: Apr. 21, 2016

(54) METHODS OF CULTURING A CELL

(71) Applicant: Alexion Pharmaceuticals, Inc., Cheshire, CT (US)

(72) Inventors: Hunter F. Malanson, Wallingford, CT (US); Pratik Jaluria, Madison, CT (US)

(21) Appl. No.: 14/882,623

(22) Filed: Oct. 14, 2015

Related U.S. Application Data
(60) Provisional application No. 62/064,442, filed on Oct. 15, 2014.

(51) Int. Cl. C12N 5/00; C07K 16/18

(52) U.S. Cl. CPC C12N 5/0018; C07K 16/18; C12N 2500/84; C07K 2317/24; C07K 2317/76

(57) ABSTRACT

Provided herein are methods of culturing a mammalian cell that include culturing a mammalian cell comprising a recombinant protein-encoding nucleic acid in a liquid culture medium containing 0.1 g/L or more processed Bovine Serum Albumin (BSA) under conditions sufficient to produce the recombinant protein, where the processed BSA is present in and/or added to the liquid culture medium prior to and/or during the culturing step.

### RELATED APPLICATIONS

This application claims priority to and the benefit of U.S. provisional patent application No. 62/064,442, filed on Oct. 15, 2014, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 25, 2015, is named AXJ-198_SL.txt and is 6,228 bytes in size.

### TECHNICAL FIELD

This invention relates to methods cell culture and the manufacture of recombinant proteins.

### BACKGROUND

Mammalian cells containing a nucleic acid that encodes a recombinant protein are often used to produce therapeutically or commercially important proteins. An example of a therapeutically important protein produced by mammalian cells is eculizumab. Eculizumab is a humanized monoclonal antibody that specifically binds to human complement component 5 (C5). Eculizumab has been approved by the FDA for treating paroxysmal nocturnal hemoglobinuria and atypical hemolytic uremic syndrome. Eculizumab is just one example of wide variety of recombinant proteins that can be produced by mammalian cell culture.

### SUMMARY

The present invention is based, at least in part, on the discovery that culturing mammalian cells in a liquid culture medium including processed bovine serum albumin (BSA) results in an increase in the viable cell density, the percentage of viable cells, and the productivity of the cells. Thus, the present specification includes methods of culturing a mammalian cell that include culturing (such as fed batch culturing or perfusion culturing) a mammalian cell containing a recombinant protein-encoding nucleic acid (such as a recombinant eculizumab-encoding nucleic acid) in a liquid culture medium including 0.1 g/L or more processed BSA under conditions sufficient to produce the recombinant protein, where the processed BSA is present in and/or added to the liquid culture medium prior to and/or during the culturing step.

Provided herein are methods of culturing a mammalian cell that include fed batch culturing a NS0 cell including a recombinant eculizumab-encoding nucleic acid in a liquid culture medium including 0.1 g/L or more processed Bovine Serum Albumin (BSA) under conditions sufficient to produce the recombinant eculizumab, where the processed BSA is present in and/or added to the liquid culture medium prior to and/or during the culturing step. In some embodiments of any of the methods described herein, the processed BSA is produced by a method comprising, consisting, or consisting essentially of the steps of: providing plasma from a bovine; desalting the plasma; filtering the plasma using ultrafiltration; precipitating euglobulin out of the plasma; filtering the plasma to remove the precipitated euglobulin; performing ion exchange chromatography on the plasma to provide an eluate including serum albumin and immunoglobulins; precipitating immunoglobulins out of the eluate using ammonium sulfate precipitation; removing the precipitated immunoglobulins from the eluate; concentrating, and freeze-drying the eluate to produce a lyophilized material including serum albumin; and optionally reconstituting the lyophilized material into a solution.

Some embodiments of any of the methods described herein further include producing the processed BSA performing the steps of: providing plasma from a bovine; desalting the plasma; filtering the plasma using ultrafiltration; precipitating euglobulin out of the plasma; filtering the plasma to remove the precipitated euglobulin; performing ion exchange chromatography on the plasma to provide an eluate including serum albumin and immunoglobulins; precipitating immunoglobulins out of the eluate using ammonium sulfate precipitation; removing the precipitated immunoglobulins from the eluate; concentrating and freeze-drying the eluate to produce a lyophilized material including serum albumin; and optionally, reconstituting the lyophilized material into a solution.

In some embodiments of any of the methods described herein, the processed BSA is produced by a method that does not include heating a solution including serum albumin, adding a stabilizer to a solution including serum albumin, or precipitating impurities out of a solution that includes a reconstituted lyophilized serum albumin. In some embodiments of any of the methods described herein, the processed BSA is a processed New Zealand BSA. In some embodiments of any of the methods described herein, the processed NZ BSA is MP Biomedical NZ Limited NZ BSA.

In some embodiments of any of the methods described herein, the liquid culture medium comprises at least 0.5 g/L processed BSA. In some embodiments of any of the methods described herein, the method includes adding processed BSA to the liquid culture medium prior to and/or during the culturing step to provide a concentration of 0.1 g/L or more processed BSA in the culture medium. In some embodiments of any of the methods described herein, the processed BSA is present in the liquid culture medium prior to the culturing step.

Some embodiments of any of the methods described herein further include collecting the recombinant eculizumab produced in the culturing step. In some embodiments of any of the methods described herein, the collecting includes lysing the NS0 cells. In some embodiments of any of the methods described herein, the recombinant eculizumab is collected from the medium.

Some embodiments of any of the methods described herein further include formulating the collected recombinant eculizumab into a pharmaceutical composition. In some embodiments of any of the methods described herein, eculizumab includes a heavy chain including or consisting of SEQ ID NO: 1 and a light chain including or consisting of SEQ ID NO: 2.

As used herein, the word “a” or “plurality” before a noun represents one or more of the particular noun. For example, the phrase “a mammalian cell” represents “one or more mammalian cells.”

The term “bovine serum albumin” or “BSA” is well known in the art. BSA is commercially available. The term “processed BSA” or “processed bovine serum albumin” refers to bovine serum albumin produced using a method described herein. Processed BSA is BSA produced by methods that exclude the steps of heating a solution that comprises the serum albumin, adding a stabilizer to a solution comprising the serum albumin, and, where lyophilized serum albumin is employed, precipitating impurities from a solution that includes the reconstituted lyophilized serum albumin. In some examples, the method used to produce processed BSA starts with providing plasma from a bovine. Non-limiting methods for producing processed BSA are described herein.

The term “processed NZ BSA” or “processed New Zealand BSA” refers to a processed BSA as defined herein that includes a serum albumin from a bovine bred and raised in New Zealand. A non-limiting example of a processed NZ BSA is MP Biomedicals NZ BSA.

The term “non-enhanced BSA” refers to a bovine serum albumin produced by a method that includes one or more of heating a solution including the serum albumin, adding a stabilizer to a solution including the serum albumin, and precipitating impurities out of a solution that includes a reconstituted lyophilized serum albumin. For example, a non-enhanced BSA is EMD Millipore Probumin®.

The term “recombinant protein” is known in the art and refers to a protein manufactured using a mammalian cell culture system. The cells may be derived from a mammalian cell, where in general, the mammalian cells in the cell culture contain an introduced nucleic acid encoding the recombinant protein of interest. The nucleic acid encoding the recombinant protein may also contain a heterologous promoter operably linked to a nucleic acid encoding the protein.

The term “production bioreactor” as used herein refers to a vessel suitable for incubating a mammalian cell culture under conditions sufficient for growth of the mammalian cells in the culture and production of a recombinant protein product by the mammalian cells in the culture. Examples of production bioreactors are known in the art.

The term “mammalian cell” refers to any cell from or derived from any mammal including, for example, a human, a hamster, a mouse, a green monkey, a rat, a pig, a cow, a hamster; or a rabbit. In some embodiments, the mammalian cell can be an immortalized cell, a differentiated cell, or an undifferentiated cell.

The term “substantially free” as used herein refers to a composition (e.g., a pharmaceutical composition) that is at least or about 90% free, or about 95%, 96%, 97%, 98%, or at least or about 99% free, or about 100% free of a specific substance (e.g., contaminating proteins from a liquid culture medium or from the lysate of a mammalian cell).

The term “culturing” or “cell culturing” as used herein refers to maintenance or growth of a mammalian cell in a liquid culture medium under a controlled set of physical conditions.

The term “liquid medium” or “liquid culture medium” refers to a fluid that contains sufficient nutrients to allow a mammalian cell to grow in the medium in vitro. For example, a liquid culture medium can include one or more of: amino acids (e.g., 20 amino acids), a purine (e.g., hypoxanthine), a pyrimidine (e.g., thymidine), choline, inositol, thiamine, folic acid, biotin, calcium, niacinamide, pyridoxine, riboflavin, thymidine, cyanocobalamin, pyruvate, lipoic acid, magnesium, glucose, sodium, potassium, iron, copper, zinc, selenium, and other necessary trace metals, and sodium bicarbonate. A liquid culture medium may include serum or serum components from a mammal. A liquid culture medium may contain trace metals, a mammalian growth hormone, and/or a mammalian growth factor. Non-limiting examples of liquid culture medium are described herein and additional examples are known in the art and are commercially available.

The term “serum-free liquid culture medium” refers to a liquid culture medium that does not contain animal serum.

“Rotary agitation” is a term well-known in the art and refers to the agitation of a culture in a bioreactor (e.g., a production bioreactor) in a generally circular fashion, e.g., clock-wise or counter-clockwise, in order to, e.g., increase the dissolved O₂ concentration in the culture in the bioreactor and/or to keep mammalian cells in suspension in the culture. Agitation can be performed using any method known in the art, e.g., an instrument that moves the culture in a circular or ellipsoidal motion, such as an impellor. Exemplary devices that can be used to perform rotary agitation are known in the art and are commercially available.

The term “immunoglobulin” refers to a polypeptide containing an amino acid sequence of at least 15 amino acids (e.g., at least 20, 30, 40, 50, 60, 70, 80, 90, or 100 amino acids, or more than 100 amino acids) of an immunoglobulin protein (e.g., a variable domain sequence, a framework sequence, or a constant domain sequence). The immunoglobulin may, for example, include at least 15 amino acids of a light chain immunoglobulin, e.g., at least 15 amino acids of a heavy chain immunoglobulin, such as a CDRH3. The immunoglobulin may be an isolated antibody (e.g., an IgG, IgE, IgD, IgA, or IgM). The immunoglobulin may be a subclass of IgG (e.g., IgG1, IgG2, IgG3, or IgG4) or may contain an Fc region that is a hybrid of two subclasses of IgG (such as a Fc region that is a hybrid of IgG2 and IgG4). The immunoglobulin may be an antibody fragment, e.g., a Fab fragment, a F(ab′)₂ fragment, or an scFv. The immunoglobulin may also be a bi-specific antibody or a tri-specific antibody, or a dimer, trimer, or multimer antibody, or a diabody, a DVD-Ig, a CODV-Ig, an Affibody®, or a Nanobody®. The immunoglobulin can also be an engineered protein containing at least one immunoglobulin domain (e.g., a fusion protein). Non-limiting examples of immunoglobulins are described herein and additional examples of immunoglobulins are known in the art.

The term “protein fragment” or “polypeptide fragment” refers to a portion of a polypeptide sequence that is at least or about 4 amino acids, at least or about 5 amino acids, at least or about 6 amino acids, at least or about 7 amino acids, at least or about 8 amino acids, at least or about 9 amino acids, at least or about 10 amino acids, at least or about 11 amino acids, at least or about 12 amino acids, at least or about 13 amino acids, at least or about 14 amino acids, at least or about 15 amino acids, at least or about 16 amino acids, at least or about 17 amino acids, at least or about 18 amino acids, at least or about 19 amino acids, or at least or about 20 amino acids in length, or more than 20 amino acids in length.

The term “engineered protein” refers to a polypeptide that is not naturally encoded by an endogenous nucleic acid present within an organism (e.g., a mammal). Examples of engineered proteins include modified enzymes with one or more amino acid substitutions, deletions, insertions, or additions that result in an increase in stability and/or catalytic activity of the engineered enzyme, fusion proteins, humanized antibodies, chimeric antibodies, divalent antibodies, trivalent antibodies, four binding domain antibodies, a diabody, and antigen-binding proteins that contain at least one recombinant scaffolding sequence.

The term “purify” or “purifying” in certain contexts means at least partially isolating a recombinant protein from one or more other components (e.g., DNA, RNA, or other proteins) present in the liquid culture medium or cell culture lysate. The extent of purification can be specified, such as at least or about 5%, e.g., at least or about 10%, 15%, 20%, 25%, 30%, 40%, 45%, 50%, 55%, 60%, 65%, 70%, 75%, 80%, 85%, 90%, or at least or about 95% to 99.9% pure by weight. Non-limiting methods for purifying a protein from a liquid culture medium or from a mammalian cell lysate are described herein and others are known in the art.

The term “secreted protein” or “secreted recombinant protein” refers to a recombinant protein that originally included a secretion signal sequence when it is translated within a mammalian cell. The signal sequence is usually removed through enzymatic cleavage in the mammalian cell and the protein is released into the extracellular space (e.g., a liquid culture medium).

The term “fed batch cell culture” or “fed batch culturing” means the incremental or continuous addition of a feed medium (e.g., liquid or solid culture medium) to an initial cell culture without substantial or significant removal of liquid culture medium from the cell culture. In some instances, the feed medium is the same as the first liquid culture medium present in the culture at the beginning of the culturing period. In other instances, the feed medium is a concentrated form of the first liquid culture medium and/or is added as a dry powder. In some examples of fed batch culture, two or more different feed media are added to the initial cell culture.

“Specific productivity rate” or “SPR” as used herein refers to the mass or enzymatic activity of a recombinant protein produced per mammalian cell per unit time (such as per day). The SPR for a recombinant antibody is usually measured as mass/cell/day. The SPR for a recombinant enzyme is usually measured as units/cell/day or (units/mass)/cell/day.

“Volume productivity rate” or “VPR” as used herein refers to the mass or enzymatic activity of recombinant protein produced per volume of culture (e.g., per L of bioreactor, vessel, or tube volume) per unit time (such as per day). The VPR for a recombinant antibody is usually measured as mass/L/day. The VPR for a recombinant enzyme is usually measured as units/L/day or mass/L/day.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a two flow charts showing the upstream and downstream processing steps used to make a non-enhanced BSA (left and center flow charts, respectively) and a flow chart showing the steps used to generate a processed BSA (right flow chart).

FIG. 2 shows the viable cell density over time in different 10,000 L-fed batch cultures of NS0 cells containing a nucleic acid that encodes eculizumab that were cultured using either (i) a first liquid culture medium containing a 1 g/L non-enhanced BSA (EMD Millipore Probumin®) and a liquid feed culture medium containing 22.1 g/L non-enhanced BSA (EMD Millipore Probumin®) (solid black lines) (n=38) or (ii) a first liquid culture medium containing 1 g/L processed BSA (MP Biomedicals NZ BSA) and a liquid feed culture medium containing 22.1 g/L processed BSA (MP Biomedicals NZ BSA) (dashed black lines) (n=21).

FIG. 3 shows the percentage cell viability over time in different 10,000 L-fed batch cultures of NS0 cells containing a nucleic acid that encodes eculizumab that were cultured using either (i) a first liquid culture medium containing a 1 g/L non-enhanced BSA (EMD Millipore Probumin®) and a liquid feed culture medium containing 22.1 g/L non-enhanced BSA (EMD Millipore Probumin®) (solid black lines) (n=38) or (ii) a first liquid culture medium containing 1 g/L processed BSA (MP Biomedicals NZ BSA) and a liquid feed culture medium containing 22.1 g/L processed BSA (MP Biomedicals NZ BSA) (dashed black lines) (n=21).

FIG. 4 shows the titer of recombinant eculizumab (mg/L) over time in different 10,000-L fed batch cultures of NS0 cells containing a nucleic acid that encodes eculizumab that were cultured using either (i) a first liquid culture medium containing a 1 g/L non-enhanced BSA (EMD Millipore Probumin®) and a liquid feed culture medium containing 22.1 g/L non-enhanced BSA (EMD Millipore Probumin®) (solid black lines) (n=38) or (ii) a first liquid culture medium containing 1 g/L processed BSA (MP Biomedicals NZ BSA) and a liquid feed culture medium containing 22.1 g/L processed BSA (MP Biomedicals NZ BSA) (dashed black lines) (n=21).

### DETAILED DESCRIPTION

Provided herein are methods of culturing a mammalian cell that include culturing (such as fed batch culturing or perfusion culturing) a mammalian cell comprising a recombinant protein-encoding nucleic acid (such as a recombinant eculizumab-encoding nucleic acid) in a liquid culture medium comprising 0.1 g/L or more processed BSA under conditions sufficient to produce the recombinant protein. In these methods, the processed BSA can be present in and/or added to the liquid culture medium (e.g., one or more of the first liquid culture medium, the feed culture medium, and the second liquid culture medium, any of the feed culture media, and/or any other liquid culture medium) prior to and/or during the culturing step. The methods described herein can achieve several benefits in the cell culture, such as one or more of increased viable cell density, increased percentage of viable cells, increased volumetric productivity rate, increased specific productivity rate, and increased recombinant protein titer, as compared to a similar cell cultured using a liquid culture medium including a non-enhanced BSA or no BSA. Non-limiting aspects of the methods of culturing a mammalian cell are described below. Any of the aspects described below can be used in any combination or with any other elements known in the art.

### Processed BSA

A processed BSA is a bovine serum albumin that is produced by the method described below. For example, a processed BSA is produced by a method that does not include heating a solution including the serum albumin, adding a stabilizer to a solution including the serum albumin, and precipitating impurities out of a solution that includes a reconstituted lyophilized serum albumin. A method for producing a processed BSA can start with a step of providing or extracting plasma from a bovine(s). A processed BSA can be a BSA produced by a method that includes, for example, the steps of providing plasma from a bovine, desalting the plasma, filtering the plasma using ultrafiltration, precipitating euglobulin out of the plasma, filtering the plasma to remove the precipitated euglobulin, performing ion exchange chromatography on the plasma to provide an eluate comprising serum albumin and immunoglobulins, precipitating immunoglobulins out of the eluate using ammonium sulfate precipitation, removing the precipitated immunoglobulins from the eluate, concentrating and freeze-drying the eluate to produce a lyophilized material comprising serum albumin, and optionally reconstituting the lyophilized material into a solution. A non-limiting example of a processed BSA is MP Biomedicals NZ BSA.

Any of the methods of culturing a mammalian cell provided herein can further include producing the processed BSA, for example, by performing the steps of providing plasma from a bovine (e.g., a bovine bred and raised in New Zealand), desalting the plasma, filtering the plasma using ultrafiltration, precipitating euglobulin out of the plasma, filtering the plasma to remove the precipitated euglobulin, performing ion exchange chromatography on the plasma to provide an eluate comprising serum albumin and immunoglobulins, precipitating immunoglobulins out of the eluate using ammonium sulfate precipitation, removing the precipitated immunoglobulins from the eluate, concentrating and freeze-drying the eluate to produce a lyophilized material including serum albumin, and optionally, reconstituting the lyophilized material into a solution.

### Culture Media Containing Processed BSA

The methods provided herein include culturing a mammalian cell containing a recombinant protein-encoding nucleic acid in a liquid culture medium (for example, a first culture medium and/or a feed culture medium) including 0.1 g/L or more (e.g., at least 0.2 g/L, at least 0.3 g/L, at least 0.4 g/L, at least 0.5 g/L, at least 0.6 g/L, at least 0.7 g/L, at least 0.8 g/L, at least 0.9 g/L, at least 1.0 g/L, at least 1.1 g/L, at least 1.2 g/L, at least 1.3 g/L, at least 1.4 g/L, at least 1.5 g/L, at least 1.6 g/L, at least 1.7 g/L, at least 1.8 g/L, at least 1.9 g/L, at least 2.0 g/L, at least 2.1 g/L, at least 2.2 g/L, at least 2.3 g/L, at least 2.4 g/L, at least 2.5 g/L, at least 2.6 g/L, at least 2.7 g/L, at least 2.8 g/L, at least 2.9 g/L, at least 3.0 g/L, at least 3.5 g/L, at least 4.0 g/L, at least 4.5 g/L, at least 5.0 g/L, at least 6.0 g/L, at least 7.0 g/L, at least 8.0 g/L, at least 9.0 g/L, at least 10.0 g/L, at least 11.0 g/L, at least 12.0 g/L, at least 13.0 g/L, at least 14.0 g/L, at least 15.0 g/L, at least 16.0 g/L, at least 17.0 g/L, at least 18.0 g/L, at least 19.0 g/L, at least 20.0 g/L, at least 25.0 g/L, at least 26.0 g/L, at least 27.0 g/L, at least 28.0 g/L, at least 29.0 g/L, or at least 30.0 g/L) processed BSA (such as NZ processed BSA). In some embodiments of any of the methods described herein, the liquid culture medium containing processed BSA can contain between about 0.1 g/L to about 30.0 g/L (e.g., between about 0.1 g/L and about 30.0 g/L, between about 0.1 g/L and about 25.0 g/L, between about 0.1 g/L and about 20.0 g/L, between about 0.1 g/L and about 15.0 g/L, between about 0.1 g/L and about 10.0 g/L, between about 0.1 g/L and about 8.0 g/L, between about 0:1 g/L and about 5.0 g/L, between about 0.1 g/L and about 4.0 g/L, between about 0.1 g/L and about 2.9 g/L, between about 0.1 g/L and about 2.8 g/L, between about 0.1 g/L and about 2.7 g/L, between about 0.1 g/L and about 2.6 g/L, between about 0.1 g/L and about 2.5 g/L, between about 0.1 g/L and about 2.4 g/L, between about 0.1 g/L and about 2.3 g/L, between about 0.1 g/L and about 2.2 g/L, between about 0.1 g/L and about 2.1 g/L, between about 0.1 g/L and about 2.0 g/L, between about 0.1 g/L and about 1.9 g/L, between about 0.1 g/L and about 1.8 g/L, between about 0.1 g/L and about 1.7 g/L, between about 0.1 g/L and about 1.6 g/L, between about 0.1 g/L and about 1.5 g/L, between about 0.1 g/L and about 1.4 g/L, between about 0.1 g/L and about 1.3 g/L, between about 0.1 g/L and about 1.2 g/L, between about 0.1 g/L and about 1.1 g/L, between about 0.1 g/L and about 1.0 g/L, between about 0.1 g/L and about 0.9 g/L, between about 0.1 g/L and about 0.8 g/L, between about 0.1 g/L and about 0.7 g/L, between about 0.1 g/L and about 0.6 g/L, between about 0.1 g/L and about 0.5 g/L, between about 0.1 g/L and about 0.4 g/L, between about 0.1 g/L and about 0.3 g/L, between about 0.5 g/L and about 30.0 g/L, between about 0.5 g/L and about 25.0 g/L, between about 0.5 g/L and about 20.0 g/L, between about 0.5 g/L and about 15.0 g/L, between about 0.5 g/L and about 10.0 g/L, between about 0.5 g/L and about 5.0 g/L, between about 0.5 g/L and about 4.0 g/L, between about 0.5 g/L and about 3.0 g/L, between about 0.5 g/L and about 2.9 g/L, between about 0.5 g/L and about 2.8 g/L, between about 0.5 g/L and about 2.7 g/L, between about 0.5 g/L and about 2.6 g/L, between about 0.5 g/L and about 2.5 g/L, between about 0.5 g/L and about 2.4 g/L, between about 0.5 g/L and about 2.3 g/L, between about 0.5 g/L and about 2.2 g/L, between about 0.5 g/L and about 2.1 g/L, between about 0.5 g/L and about 2.0 g/L, between about 0.5 g/L and about 1.9 g/L, between about 0.5 g/L and about 1.8 g/L, between about 0.5 g/L and about 1.7 g/L, between about 0.5 g/L and about 1.6 g/L, between about 0.5 g/L and about 1.5 g/L, between about 0.5 g/L and about 1.4 g/L, between about 0.5 g/L and about 1.3 g/L, between about 0.5 g/L and about 1.2 g/L, between about 0.5 g/L and about 1.1 g/L, between about 0.5 g/L and about 1.0 g/L, between about 0.5 g/L and about 0.9 g/L, between about 0.5 g/L and about 0.8 g/L, between about 0.5 g/L and about 0.7 g/L, between about 1.0 g/L and about 3.0 g/L, between about 1.0 g/L and about 30.0 g/L, between about 1.0 g/L and about 25.0 g/L, between about 1.0 g/L and about 20.0 g/L, between about 1.0 g/L and about 15.0 g/L, between about 1.0 g/L an about 10.0 g/L, between about 1.0 g/L and about 5.0 g/L, between about 1.0 g/L and about 2.9 g/L, between about 1.0 g/L and about 2.8 g/L, between about 1.0 g/L and about 2.7 g/L, between about 1.0 g/L and about 2.6 g/L, between about 1.0 g/L and about 2.5 g/L, between about 1.0 g/L and about 2.4 g/L, between about 1.0 g/L and about 2.3 g/L, between about 1.0 g/L and about 2.2 g/L, between about 1.0 g/L and about 2.1 g/L, between about 1.0 g/L and about 2.0 g/L, between about 1.0 g/L and about 1.9 g/L, between about 1.0 g/L and about 1.8 g/L, between about 1.0 g/L and about 1.7 g/L, between about 1.0 g/L and about 1.6 g/L, between about 1.0 g/L and about 1.5 g/L, between about 1.0 g/L and about 1.4 g/L, between about 1.0 g/L and about 1.3 g/L, between about 1.0 g/L and about 1.2 g/L, between about 1.5 g/L and about 30 g/L, between about 1.5 g/L and about 25 g/L, between about 1.5 g/L and about 20 g/L, between about 1.5 g/L and about 15 g/L, between about 1.5 g/L and about 10 g/L, between about 1.5 g/L and about 5.0 g/L, between about 1.5 g/L and about 3.0 g/L, between about 1.5 g/L and about 2.9 g/L, between about 1.5 g/L and about 2.8 g/L, between about 1.5 g/L and about 2.7 g/L, between about 1.5 g/L and about 2.6 g/L, between about 1.5 g/L and about 2.5 g/L, between about 1.5 g/L and about 2.4 g/L, between about 1.5 g/L and about 2.3 g/L, between about 1.5 g/L and about 2.2 g/L, between about 1.5 g/L and about 2.1 g/L, between about 0.1.5 g/L and about 2.0 g/L, between about 1.5 g/L and about 1.9 g/L, between about 1.5 g/L and about 1.8 g/L, between about 1.5 g/L and about 1.7 g/L, between about 2.0 g/L and about 30 g/L, between about 2.0 g/L and about 25 g/L, between about 2.0 g/L and about 20 g/L, between about 2.0 g/L and about 15 g/L, between about 2.0 g/L and about 10 g/L, between about 2.0 g/L and about 5 g/L, between about 2.0 g/L and about 3.0 g/L, between about 2.0 g/L and about 2.9 g/L, between about 2.0 g/L and about 2.8 g/L, between about 2.0 g/L and about 2.7 g/L, between about 2.0 g/L and about 2.6 g/L, between about 2.0 g/L and about 2.5 g/L, between about 2.0 g/L and about 2.4 g/L, between about 2.0 g/L and about 2.3 g/L, between about 2.0 g/L and about 2.2 g/L, between about 2.5 g/L and about 30 g/L, between about 2.5 g/L and about 25 g/L, between about 2.5 g/L and about 20 g/L, between about 2.5 g/L and about 15 g/L, between about 2.5 g/L and about 10 g/L, between about 2.5 g/L and about 5 g/L, between about 2.5 g/L and about 3.0 g/L, between about 2.5 g/L and about 2.9 g/L, between about 2.5 g/L and about 2.8 g/L, between about 2.5 g/L and about 2.7 g/L, between about 5.0 g/L and about 30 g/L, between about 5.0 g/L and about 25.0 g/L, between about 5.0 g/L and about 20.0 g/L, between about 5.0 g/L and about 15.0 g/L, between about 5.0 g/L and about 10.0 g/L, between about 10.0 g/L and about 30 g/L, between about 10.0 g/L and about 25.0 g/L, between about 10.0 g/L and about 20.0 g/L, between about 10.0 g/L and about 15.0 g/L, between about 15.0 g/L and about 30 g/L, between about 15.0 g/L and about 25.0 g/L, between about 15.0 g/L and about 20.0 g/L, between about 20.0 g/L and about 30.0 g/L, between about 20.0 g/L and about 25.0 g/L, or between about 21 g/L and about 23 g/L) processed BSA.

The liquid culture medium including 0.1 g/L or more processed BSA can the first liquid culture medium or can be both the first liquid culture feed medium in methods that include fed batch culturing of the mammalian cell. Fed batch culturing in any of the methods described herein can include the use of two different liquid culture feed media, with one or both of the two different liquid culture feed media including 0.1 g/L or more processed BSA. The liquid culture medium including 0.1 g/L or more processed BSA can be one or both of the first liquid culture feed medium and the second liquid culture feed medium in methods that include perfusion culturing of the mammalian cell.

As is well-known in the art, a liquid culture medium containing processed BSA can be produced by adding a sufficient amount of processed BSA (in solid form or as a solution) to a liquid culture medium to achieve a desired final concentration of processed BSA in the liquid culture medium.

In some methods, the concentration of processed BSA present in the culture can remain substantially constant throughout the culturing period. In other methods, the concentration of processed BSA present in the culture can increase during the culturing period. In some embodiments, the processed BSA may be present in the liquid culture medium at the start of the culturing period (the first liquid culture medium). In other embodiments, the processed BSA may be added to the first liquid culture medium after the start of the culturing period (e.g., by bolus injection or by adding a feed culture medium or a second liquid culture medium that contains a processed BSA).

### Culturing Mammalian Cells

Provided herein are methods of culturing a mammalian cell that include culturing a mammalian cell (e.g., a NS0 cell) containing a recombinant protein-encoding nucleic acid (e.g., a recombinant eculizumab-encoding nucleic acid) in a liquid culture medium including 0.1 g/L or more processed BSA. In some embodiments of the methods, the processed BSA is present in and/or added to the liquid culture medium prior to and/or during culturing. For example, the processed BSA can be present in the first liquid culture medium prior to culturing. In some embodiments, the processed BSA is actively added to the first liquid culture medium prior to culturing. In some examples, the processed BSA is added to the first liquid culture medium during culturing (e.g., by bolus injection of processed BSA, or by adding a feed culture medium or a second liquid culture medium containing processed BSA). Any of the non-limiting liquid culture media containing a processed BSA described herein can be used in any of the methods described herein.

In some embodiments of the culturing methods, the culturing is fed batch culturing. In other embodiments of the culturing methods, the culturing is perfusion culturing. In any of the culturing methods described herein, the cell can be a NS0 cell containing a recombinant eculizumab-encoding nucleic acid. Non-limiting examples of the aspects of culturing mammalian cells that can be used in the methods described herein are described below. As can be appreciated, any of the aspects described below or additional aspects known in the art, can be used in any combination.

Fed Batch Culturing

The culturing step in the methods described herein can include fed batch culturing. As is known in the art, fed batch culturing includes the incremental (periodic) or continuous addition of a feed culture medium to an initial cell culture without substantial or significant removal of the first liquid culture medium from the cell culture. The cell culture in fed batch culturing can be disposed in a bioreactor (e.g., a production bioreactor, such as a 10,000-L production bioreactor). In some instances, the feed culture medium is the same as the first liquid culture medium. The feed culture medium may be either in a liquid form or a dry powder. In other instances, the feed culture medium is a concentrated form of the first liquid culture medium and/or is added as a dry powder. In some embodiments, both a first liquid feed culture medium and a different second liquid feed culture medium are added (e.g., continuously added) to the first liquid culture medium. In some examples, the addition of the first liquid feed culture medium and addition of the second liquid feed culture medium to the culture is initiated at about the same time. In some examples, the total volume of the first liquid feed culture medium and the second liquid feed culture medium added to the culture over the entire culturing period are about the same.

When the feed culture medium is added continuously, the rate of addition of the feed culture medium can be held constant or can be increased (e.g., steadily increased) over the culturing period. A continuous addition of feed culture medium can start at a specific time point during the culturing period (e.g., when the mammalian cells reach a target viable cell density, e.g., a viable cell density of about 1×10⁶ cells/mL, about 1.1×10⁶ cells/mL, about 1.2×10⁶ cells/mL, about 1.3×10⁶ cells/mL, about 1.4×10⁶ cells/mL, about 1.5×10⁶ cells/mL, about 1.6×10⁶ cells/mL, about 1.7×10⁶ cells/mL, about 1.8×10⁶ cells/mL, about 1.9×10⁶ cells/mL, or about 2.0×10⁶ cells/mL). In some embodiments, the continuous addition of feed culture medium can be initiated at day 2, day 3, day 4, or day 5 of the culturing period.

In some embodiments, an incremental (periodic) addition of feed culture medium can begin when the mammalian cells reach a target cell density (e.g., about 1×10⁶ cells/mL, about 1.1×10⁶ cells/mL, about 1.2×10⁶ cells/mL, about 1.3×10⁶ cells/mL, about 1.4×10⁶ cells/mL, about 1.5×10⁶ cells/mL, about 1.6×10⁶ cells/mL, about 1.7×10⁶ cells/mL, about 1.8×10⁶ cells/mL, about 1.9×10⁶, or about 2.0×10⁶ cells/mL). Incremental feed culture media addition can occur at regular intervals (e.g., every day, every other day, or every third day) or can occur when the cells reach specific target cell densities (e.g., target cell densities that increase over the culturing period). In some embodiments, the amount of feed culture medium added can progressively increase between the first incremental addition of feed culture medium and subsequent additions (of feed culture medium. The volume of a liquid culture feed culture medium added to the initial cell culture over any 24 hour period in the culturing period can be some fraction of the initial volume of the bioreactor containing the culture or some fraction of the volume of the initial culture.

For example, the addition of the liquid feed culture medium (continuously or periodically) can occur at a time point that is between 6 hours and 7 days, between about 6 hours and about 6 days, between about 6 hours and about 5 days, between about 6 hours and about 4 days, between about 6 hours and about 3 days, between about 6 hours and about 2 days, between about 6 hours and about 1 day, between about 12 hours and about 7 days, between about 12 hours and about 6 days, between about 12 hours and about 5 days, between about 12 hours and about 4 days, between about 12 hours and about 3 days, between about 12 hours and about 2 days, between about 1 day and about 7 days, between about 1 day and about 6 days, between about 1 day and about 5 days, between about 1 day and about 4 days, between about 1 day and about 3 days, between about 1 day and about 2 days, between about 2 days and about 7 days, between about 2 days and about 6 days, between about 2 days and about 5 days, between about 2 days and about 4 days, between about 2 days and about 3 days, between about 3 days and about 7 days, between about 3 days and about 6 days, between about 3 days and about 5 days, between about 3 days and about 4 days, between about 4 days and about 7 days, between about 4 days and about 6 days, between about 4 days and about 5 days, between about 5 days and about 7 days, or between about 5 days and about 6 days, after the start of the culturing period.

The volume of a liquid feed culture medium added (continuously or periodically) to the initial cell culture over any 24 hour period can be between 0.01× and about 0.3× of the capacity of the bioreactor. The fraction may be between about 0.01× and about 0.28×, between about 0.01× and about 0.26×, between about 0.01× and about 0.24×, between about 0.01× and about 0.22×, between about 0.01× and about 0.20×, between about 0.01× and about 0.18×, between about 0.01× and about 0.16×, between about 0.01× and about 0.14×, between about 0.01× and about 0.12×, between about 0.01× and about 0.10×, between about 0.01× and about 0.08×, between about 0.01× and about 0.06×, between about 0.01× and about 0.04×, between about 0.02× and about 0.3×, between about 0.02× and about 0.28×, between about 0.02× and about 0.26×, between about 0.02× and about 0.24×, between about 0.02× and about 0.22×, between about 0.02× and about 0.20×, between about 0.02× and about 0.18×, between about 0.02× and about 0.16×, between about 0.02× and about 0.14×, between about 0.02× and about 0.12×, between about 0.02× and about 0.10×, between about 0.02× and about 0.08×, between about 0.02× and about 0.06×, between about 0.02× and about 0.05×, between about 0.02× and about 0.04×, between about 0.02× and about 0.03×, between about 0.025× and about 0.3×, between about 0.025× and about 0.28×, between about 0.025× and about 0.26×, between about 0.025× and about 0.24×, between about 0.025× and about 0.22×, between about 0.025× and about 0.20×, between about 0.025× and about 0.18×, between about 0.025× and about 0.16×, between about 0.025× and about 0.14×, between about 0.025× and about 0.12×, between about 0.025× and about 0.10×, between about 0.025× and about 0.08×, between about 0.025× and about 0.06×, between about 0.025× and about 0.04×, between about 0.05× and about 0.3×, between about 0.05× and about 0.28×, between about 0.05× and about 0.26×, between about 0.05× and about 0.24×, between about 0.05× and about 0.22×, between about 0.05× and about 0.20×, between about 0.05× and about 0.18×, between about 0.05× and about 0.16×, between about 0.05× and about 0.14×, between about 0.05× and about 0.12×, between about 0.05× and about 0.10×, between about 0.1× and about 0.3×, between about 0.1× and about 0.28×, between about 0.1× and about 0.26×, between about 0.1× and about 0.24×, between about 0.1× and about 0.22×, between about 0.1× and about 0.20×, between about 0.1× and about 0.18×, between about 0.1× and about 0.16× between about 0.1× and about 0.14×, between about 0.1×, between about 0.15× and about 0.3×, between about 0.15× and about 0.2×, between about 0.2× and about 0.3×, or between about 0.25× and about 0.3×, of the capacity of the bioreactor.

In other embodiments, the volume of a liquid feed culture medium added (continuously or periodically) to the initial cell culture over any 24 hour period during the culturing period can be between 0.02× and about 1.0×, between about 0.02× and about 0.9×, between about 0.02× and about 0.8×, between about 0.02× and about 0.7×, between about 0.02× and about 0.6×, between about 0.02× and about 0.5×, between about 0.02× and about 0.4×, between about 0.02× and about 0.3×, between about 0.02× and about 0.2×, between about 0.02× and about 0.1×, between about 0.02× and about 0.08×, between about 0.02× and about 0.06×, between about 0.02× and about 0.05×, between about 0.02× and about 0.04×, between about 0.02× and about 0.03×, between about 0.05× and about 1.0×, between about 0.05× and about 0.8×, between about 0.05× and about 0.7×, between about 0.05× and about 0.6×, between about 0.05× and about 0.5×, between about 0.05× and about 0.4×, between about 0.05× and about 0.3×, between about 0.05× and about 0.2×, between about 0.05× and about 0.1×, between about 0.1× and about 1.0×, between about 0.1× and about 0.9×, between about 0.1× and about 0.8×, between about 0.1× and about 0.7×, between about 0.1× and about 0.6×, between about 0.1× and about 0.5×, between about 0.1× and about 0.4×, between about 0.1× and about 0.3×, between about 0.1× and about 0.2×, between about 0.2× and about 1.0×, between about 0.2× and about 0.9×, between about 0.2× and about 0.8×, between about 0.2× and about 0.7×, between about 0.2× and about 0.6×, between about 0.2× and about 0.5×, or between about 0.2× and about 0.4× of the volume of the initial cell culture.

The total amount of feed culture medium added (continuously or periodically) over the entire culturing period can be between about 1% and about 40% (e.g., between about 1% and about 35%, between about 1% and about 30%, between about 1% and about 25%, between about 1% and about 20%, between about 1% and about 15%, between about 1% and about 10%, between about 1% and about 5%, between about 1% and about 4%, between about 2% and about 40%, between about 2% and about 35%, between about 2% and about 30%, between about 2% and about 25%, between about 2% and about 20%, between about 2% and about 15%, between about 2% and about 10%, between about 2% and about 5%, between about 3% and about 40%, between about 3% and about 35%, between about 3% and about 30%, between about 3% and about 25%, between about 3% and about 20%, between about 3% and about 15%, between about 3% and about 10%, between about 3% and about 5%, between about 4% and about 40%, between about 4% and about 35%, between about 4% and about 30%, between about 4% and about 25%, between about 4% and about 20%, between about 4% and about 15%, between about 4% and about 10%, between about 4% and about 8%, between about 5% and about 40%, between about 5% and about 35%, between about 5% and about 30%, between about 5% and about 25%, between about 5% and about 20%, between about 5% and about 15%, between about 5% and about 10%, between about 10% and about 40%, between about 10% and about 35%, between about 10% and about 30%, between about 10% and about 25%, between about 10% and about 20%, between about 10% and about 15%, between about 15% and about 40%, between about 15% and about 35%, between about 15% and about 30%, between about 15% and about 25%, between about 15% and about 20%, between about 20% and about 40%, between about 20% and about 35%, between about 20% and about 30%, between about 20% and about 25%, between about 25% and about 40%, between about 25% and about 35%, between about 25% and about 30%, between about 30% and about 40%, between about 30% and about 35%, or between about 35% and about 40%) of the volume of the initial culture.

In some examples, two different feed culture media are added (continuously or incrementally) during feed batch culturing. The amount or volume of the first feed culture medium and the second feed culture medium added can be substantially the same or can differ. The first feed culture medium can be in the form of a liquid and the second feed culture medium can be in the form of a solid. The first feed culture medium and the second feed culture medium can be liquid feed culture media.

Perfusion Culturing

The culturing step in the methods described herein can be perfusion culturing. As is known in the art, perfusion culturing includes removing from a bioreactor (e.g., a production bioreactor) a first volume of a first liquid culture medium, and adding to the production bioreactor a second volume of a second liquid culture medium, wherein the first volume and the second volume are about equal. The mammalian cells are retained in the bioreactor by some cell retention device or through techniques, such as cell settling in a settling cone. The removal and addition of culture media in perfusion culturing can be performed simultaneously or sequentially, or some combination of the two. Further, removal and addition can be performed continuously, such as at a rate that removes and replaces a volume of between 0.1% to 800%, between 1% and 700%, between 1% and 600%, between 1% and 500%, between 1% and 400%, between 1% and 350%, between 1% and 300%, between 1% and 250%, between 1% and 100%, between 100% and 200%, between 5% and 150%, between 10% and 50%, between 15% and 40%, between 8% and 80%, or between 4% and 30% of the capacity of the bioreactor.

The first volume of the first liquid culture medium removed and the second volume of the second liquid culture medium added can in some instances be held approximately the same over each 24-hour period. As is known in the art, the rate at which the first volume of the first liquid culture medium is removed (volume/unit of time) and the rate at which the second volume of the second liquid culture medium is added (volume/unit of time) can be varied and, depends on the conditions of the particular cell culture system. The rate at which the first volume of the first liquid culture medium is removed (volume/unit of time) and the rate at which the second volume of the second liquid culture medium is added (volume/unit of time) can be about the same or can be different.

Alternatively, the volume removed and added can change by gradually increasing over each 24-hour period. For example, the volume of the first liquid culture medium removed and the volume of the second liquid culture medium added within each 24-hour period can be increased over the culturing period. The volume can be increased a volume that is between 0.5% to about 20% of the capacity of the bioreactor over a 24-hour period. The volume can be increased over the culturing period to a volume that is about 25% to about 150% of the capacity of the bioreactor or the first liquid culture medium volume over a 24-hour period.

In some examples of the methods described herein, after the first 48 to 96 hours of the culturing period, in each 24-hour period, the first volume of the first liquid culture medium removed and the second volume of the second liquid culture medium added is about 10% to about 95%, about 10% to about 20%, about 20% to about 30%, about 30% to about 40%, about 40% to about 50%, about 50% to about 60%, about 60% to about 70%, about 70% to about 80%, about 80% to about 90%, about 85% to about 95%, about 60% to about 80%, or about 70% of the volume of the first liquid culture medium.

Skilled practitioners will appreciate that the first liquid culture medium and the second liquid culture medium can be the same type of media. In other instances, the first liquid culture medium and the second liquid culture medium can be different. The second liquid culture medium may be more concentrated with respect to one or more media components. In some embodiments, the first liquid culture medium contains processed BSA, the second liquid culture medium contains processed BSA, or both the first and the second liquid culture medium contains processed BSA.

The first volume of the first liquid culture medium can be removed by using any automated system. For example alternating tangential flow filtration may be used. Alternatively, the first volume of the first liquid culture medium can be removed by seeping or gravity flow of the first volume of the first liquid culture medium through a sterile membrane with a molecular weight cut-off that excludes the mammalian cell. Alternatively, the first volume of the first liquid culture medium can be removed by stopping or significantly decreasing the rate of agitation for a period of at least 1 minute, at least 2 minutes, 3 minutes, 4 minutes, 5 minutes, 10 minutes, 15 minutes, 20 minutes, 25 minutes, 30 minutes, 40 minutes, 50 minutes, or 1 hour, and removing or aspirating the first volume of the first liquid culture medium from the top of the production bioreactor.

The second volume of the second liquid culture medium can be added to the first liquid culture medium by a pump. The second liquid culture medium can be added to the first liquid medium manually, such as by pipetting or injecting the second volume of the second liquid culture medium directly onto the first liquid culture medium or in an automated fashion.

Mammalian Cells

The mammalian cells that are cultured in the methods described herein can be a variety of different mammalian cells. In some examples, the mammalian cell is an adherent cell and the cell culture includes microcarriers. In some examples, the mammalian cell can be a cell that grows in suspension. Non-limiting examples of mammalian cells that can be cultured using the methods described herein include: Chinese hamster ovary (CHO) cells (e.g., CHO DG44 cells, CHO-K1 cells), Sp2/0 myeloma cells, other myeloma cells, such as NS0 cells, B-cells, hybridoma cells; T-cells, human embryonic kidney (HEK) cells (e.g, HEK 293E and HEK 293F), African green monkey kidney epithelial cells (Vero) cells, and Madin-Darby Canine (Cocker Spaniel) kidney epithelial cells (MDCK) cells. Additional mammalian cells that can be cultured using the methods described herein are known in the art. In non-limiting examples of any of the methods described herein, the cell density of mammalian cells present in the production bioreactor at the start of the culturing period (initial cell density) is about 0.1×10⁶ cells/mL to about 0.5×10⁶ cells/mL, about 0.2×10⁶ cells/mL to about 0.4×10⁶ cells/mL, or about 0.25×10⁶ cells/mL to about 0.5×10⁶ cells/mL.

The mammalian cells used in the methods described herein can contain a recombinant nucleic acid that is stably integrated in the mammalian cell's genome and encodes a recombinant protein. In some embodiments, the recombinant protein is secreted by the mammalian cell into the liquid culture medium. In some instances, the cultured mammalian cells are derived from a seed culture. More particularly, the initial cell culture is the result of a seed train process or a culture from another bioreactor.

Culture Media

Liquid culture media that can be used in the culturing step are known in the art. The liquid culture medium can be a serum-free liquid culture medium. Non-limiting examples of culture media, including serum-free culture media, are commercially available.

A liquid culture medium typically contains an energy source from a carbohydrate, such as glucose, amino acids (e.g., the basic set of twenty amino acids plus cysteine), vitamins, free fatty acids, trace elements, and other organic compounds required at low concentrations. The liquid culture medium can be supplemented with salts and buffers (e.g., calcium, magnesium, and phosphate salts), nucleosides and bases (e.g., adenosine, thymidine, and hypoxanthine), protein and tissue hydrolysates, and/or any combination of these or other additives.

Non-limiting examples of liquid culture media that can be useful in the presently described methods include, e.g., CD CHO, Opti CHO, and Forti CHO (all available from Life Technologies; Grand Island, N.Y.), Hycell CHO medium (Thermo Fisher Scientific, Inc.; Waltham, Mass.), Ex-cell CD CHO Fusion medium (Sigma-Aldrich Co.; St. Louis, Mo.), and PowerCHO medium (Lonza Group, Ltd.; Basel, Switzerland). Medium components that also may be useful in the present methods include, but are not limited to, chemically-defined (CD) hydrolysates, e.g., CD peptone, CD polypeptides (two or more amino acids), and CD growth factors. Additional examples of liquid culture medium and medium components are known in the art.

The culture media used in the culturing of the mammalian cells can have a pH of between about 6.5 and about 7.5, between about 6.5 and about 7.4, between about 6.5 and about 7.3, between about 6.5 and about 7.2, between about 6.5 and about 7.1, between about 6.5 and about 7.0, between about 6.5 and about 6.9, between about 6.5 and about 6.8, between about 6.5 and about 6.7, between about 6.6 and about 7.5, between about 6.6 and about 7.4, between about 6.6 and about 7.3, between about 6.6 and about 7.2, between about 6.6 and about 7.1, between about 6.6 and about 7.0, between about 6.6 and about 6.9, between about 6.6 and about 6.8, between about 6.7 and about 7.5, between about 6.7 and about 7.4, between about 6.7 and about 7.3, between about 6.7 and about 7.2, between about 6.7 and about 7.1, between about 6.7 and about 7.0, between about 6.7 and about 6.9, between about 6.8 and about 7.5, between about 6.8 and about 7.4, between about 6.8 and about 7.3, between about 6.8 and about 7.2, between about 6.8 and about 7.1, between about 6.8 and about 7.0, between about 6.9 and about 7.5, between about 6.9 and about 7.4, between about 6.9 and about 7.3, between about 6.9 and about 7.2, between about 6.9 and about 7.1, between about 7.0 and about 7.5, between about 7.0 and about 7.4, between about 7.0 and about 7.3, between about 7.0 and about 7.2, between about 7.1 and about 7.5, between about 7.1 and about 7.4, between about 7.1 and about 7.3, between about 7.2 and about 7.5, between about 7.2 and about 7.4, or between about 7.3 and about 7.5.

Skilled practitioners will appreciate that the liquid culture medium used in culturing can be the same or can change during the culturing period depending on cell culture conditions.

In some embodiments, the feed culture media added in a fed batch culture can be a solid composition. Examples of solid feed culture media that can be added to a fed batch culture are known in the art.

Agitation

The culturing of a mammalian cell usually includes some form of agitation for mixing of the culture. For example, the agitation used in culturing can be rotary agitation using an impeller. The agitation can occur at a frequency of at about 25 RPM to about 500 RPM, between about 25 RPM and about 480 RPM, between about 25 RPM and about 460 RPM, between about 25 RPM and about 440 RPM, between about 25 RPM and about 420 RPM, between about 25 RPM and about 400 RPM, between about 25 RPM and about 380 RPM, between about 25 RPM and about 360 RPM, between about 25 RPM and about 340 RPM, between about 25 RPM and about 320 RPM, between about 25 RPM and about 300 RPM, between about 25 RPM and about 280 RPM, between about 25 RPM and about 260 RPM, between about 25 RPM and about 240 RPM, between about 25 RPM and about 220 RPM, between about 25 RPM and about 200 RPM, between about 25 RPM and about 180 RPM, between about 25 RPM and about 160 RPM, between about 25 RPM and about 140 RPM, between about 25 RPM and about 120 RPM, between about 25 RPM and about 100 RPM, between about 25 RPM and about 80 RPM, between about 25 RPM and about 60 RPM, between about 25 RPM and about 40 RPM, between about 25 RPM and about 35 RPM, between about 25 RPM and about 30 RPM, between about 50 RPM to about 500 RPM, between about 50 RPM and about 480 RPM, between about 50 RPM and about 460 RPM, between about 50 RPM and about 440 RPM, between about 50 RPM and about 420 RPM, between about 50 RPM and about 400 RPM, between about 50 RPM and about 380 RPM, between about 50 RPM and about 360 RPM, between about 50 RPM and about 340 RPM, between about 50 RPM and about 320 RPM, between about 50 RPM and about 300 RPM, between about 50 RPM and about 280 RPM, between about 50 RPM and about 260 RPM, between about 50 RPM and about 240 RPM, between about 50 RPM and about 220 RPM, between about 50 RPM and about 200 RPM, between about 50 RPM and about 180 RPM, between about 50 RPM and about 160 RPM, between about 50 RPM and about 140 RPM, between about 50 RPM and about 120 RPM, between about 50 RPM and about 100 RPM, between about 50 RPM and about 80 RPM, between about 50 RPM and about 60 RPM, between about 75 RPM to about 500 RPM, between about 75 RPM and about 480 RPM, between about 75 RPM and about 460 RPM, between about 75 RPM and about 440 RPM, between about 75 RPM and about 420 RPM, between about 75 RPM and about 400 RPM, between about 75 RPM and about 380 RPM, between about 75 RPM and about 360 RPM, between about 75 RPM and about 340 RPM, between about 75 RPM and about 320 RPM, between about 75 RPM and about 300 RPM, between about 75 RPM and about 280 RPM, between about 75 RPM and about 260 RPM, between about 75 RPM and about 240 RPM, between about 75 RPM and about 220 RPM, between about 75 RPM and about 200 RPM, between about 75 RPM and about 180 RPM, between about 75 RPM and about 160 RPM, between about 75 RPM and about 140 RPM, between about 75 RPM and about 120 RPM, between about 75 RPM and about 100 RPM, between about 75 RPM and about 80 RPM, between about 100 RPM to about 500 RPM, between about 100 RPM and about 480 RPM, between about 100 RPM and about 460 RPM, between about 100 RPM and about 440 RPM, between about 100 RPM and about 420 RPM, between about 100 RPM and about 400 RPM, between about 100 RPM and about 380 RPM, between about 100 RPM and about 360 RPM, between about 100 RPM and about 340 RPM, between about 100 RPM and about 320 RPM, between about 100 RPM and about 300 RPM, between about 100 RPM and about 280 RPM, between about 100 RPM and about 260 RPM, between about 100 RPM and about 240 RPM, between about 100 RPM and about 220 RPM, between about 100 RPM and about 200 RPM, between about 100 RPM and about 180 RPM, between about 100 RPM and about 160 RPM, between about 100 RPM and about 140 RPM, between about 100 RPM and about 120 RPM, between about 150 RPM to about 500 RPM, between about 150 RPM and about 480 RPM, between about 150 RPM and about 460 RPM, between about 150 RPM and about 440 RPM, between about 150 RPM and about 420 RPM, between about 150 RPM and about 400 RPM, between about 150 RPM and about 380 RPM, between about 150 RPM and about 360 RPM, between about 150 RPM and about 340 RPM, between about 150 RPM and about 320 RPM, between about 150 RPM and about 300 RPM, between about 150 RPM and about 280 RPM, between about 150 RPM and about 260 RPM, between about 150 RPM and about 240 RPM, between about 150 RPM and about 220 RPM, between about 150 RPM and about 200 RPM, between about 150 RPM and about 180 RPM, between about 150 RPM and about 160 RPM, between at about 200 RPM to about 500 RPM, between about 200 RPM and 480 RPM, between about 200 RPM and about 460 RPM, between about 200 RPM and about 440 RPM, between about 200 RPM and about 420 RPM, between about 200 RPM and about 400 RPM, between about 200 RPM and about 380 RPM, between about 200 RPM and about 360 RPM, between about 200 RPM and about 340 RPM, between about 200 RPM and about 320 RPM, between about 200 RPM and about 300 RPM, between about 200 RPM and about 280 RPM, between about 200 RPM and about 260 RPM, between about 200 RPM and about 240 RPM, between about 200 RPM and about 220 RPM, between about 240 RPM and about 500 RPM, between about 240 RPM and about 480 RPM, between about 240 RPM and about 460 RPM, between about 240 RPM and about 440 RPM, between about 240 RPM and about 420 RPM, between about 240 RPM and about 400 RPM, between about 240 RPM and about 380 RPM, between about 240 RPM and about 360 RPM, between about 240 RPM and about 340 RPM, between about 240 RPM and about 320 RPM, between about 240 RPM and about 300 RPM, between about 240 RPM and about 280 RPM, between about 240 RPM and about 260 RPM, between about 260 RPM and about 500 RPM, between about 260 RPM and about 480 RPM, between about 260 RPM and about 460 RPM, between about 260 RPM and about 440 RPM, between about 260 RPM and about 420 RPM, between about 260 RPM and about 400 RPM, between about 260 RPM and about 380 RPM, between about 260 RPM and about 360 RPM, between about 260 RPM and about 340 RPM, between about 260 RPM and about 320 RPM, between about 260 RPM and about 300 RPM, between about 260 RPM and about 280 RPM, between about 280 RPM and about 500 RPM, between about 280 RPM and about 480 RPM, between about 280 RPM and about 460 RPM, between about 280 RPM and about 440 RPM, between about 280 RPM and about 420 RPM, between about 280 RPM and about 400 RPM, between about 280 RPM and about 380 RPM, between about 280 RPM and about 360 RPM, between about 280 RPM and about 340 RPM, between about 280 RPM and about 320 RPM, between about 280 RPM and about 280 RPM, between about 300 RPM and about 500 RPM, between about 380 RPM and about 480 RPM, between about 380 RPM and about 460 RPM, between about 380 RPM and about 440 RPM, between about 380 RPM and about 420 RPM, between about 380 RPM and about 400 RPM, between about 400 RPM and about 500 RPM, between about 400 RPM and about 480 RPM, between about 400 RPM and about 460 RPM, between about 400 RPM and about 440 RPM, or between about 400 RPM and about 420 RPM. The agitation can be performed continuously or periodically.

Temperature

The culturing step described herein can be performed at a temperature of 32° C. to about 39° C., about 32° C. to about 37° C., between about 32° C. and about 37.5° C., between about 34° C. and about 37° C., between about 35° C. and about 37° C., between about 35.5° C. and about 37.5° C., between about 36° C. and about 37° C., or about 36.5° C. For example, the mammalian cells can be incubated at a temperature of about 37° C. from the beginning to the end of the culturing period. Skilled practitioners will appreciate that the temperature can be changed or may vary slightly during the culturing period, e.g., on an hourly or daily basis. For example, the temperature can be changed or shifted (e.g., increased or decreased) at about one day, two days, three days, four days, five days, six days, seven days, eight days, nine days, ten days, eleven days, twelve days, fourteen days, or fifteen days after the start of the culturing period, or at any time point within the culturing period. For example, the temperature can be shifted upwards by about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0° C. In another example, the temperature can be shifted downwards by about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10° C.

CO₂

The culturing step can be performed using an atmosphere containing about 1% to 15% CO₂, at most or about 14% CO₂, 12% CO₂, 10% CO₂, 8% CO₂, 6% CO₂, 5% CO₂, 4% CO₂, 3% CO₂, 2% CO₂, or at most or about 1% CO₂. Methods for sparging CO2 into a production bioreactor are well known in the art.

Any of the methods described herein can also include culturing the cells during the first time period in a humidified atmosphere comprising at least or about 20%, 30%, 40%, 50%, 60%, 70%, 85%, 80%, 85%, 90%, or at least or about 95% humidity, or about 100% humidity.

dO₂

The culturing step can be performed by maintaining a dissolved oxygen (dO₂) in the cell culture of between about 3% and about 55%, between about 3% and about 50%, between about 3% and about 45%, between about 3% and about 40%, between about 3% and about 35%, between about 3% and about 30%, between about 3% and about 25%, between about 3% and about 20%, between about 3% and about 15%, between about 5% and about 55%, between about 5% and about 50%, between about 5% and about 45%, between about 5% and about 40%, between about 5% and about 35%, between about 5% and about 30%, between about 5% and about 25%, between about 5% and about 20%, between about 5% and about 15%, between about 5% and about 10%, between about 10% and about 55%, between about 10% and about 50%, between about 10% and about 45%, between about 10% and about 40%, between about 10% and about 35%, between about 10% and about 30%, between about 10% and about 25%, between about 10% and about 20%, between about 15% and about 55%, between about 15% and about 50%, between about 15% and about 45%, between about 15% and about 40%, between about 15% and about 35%, between about 15% and about 30%, between about 15% and about 25%, between about 15% and about 20%, between about 20% and about 55%, between about 20% and about 50%, between about 20% and about 45%, between about 20% and about 40%, between about 20% and about 35%, between about 20% and about 30%, between about 20% and about 25%, between about 25% and about 55%, between about 25% and about 50%, between about 25% and about 45%, between about 25% and about 40%, between about 25% and about 35%, between about 25% and about 30%, between about 30% and about 55%, between about 30% and about 50%, between about 30% and about 45%, between about 30% and about 40%, between about 30% and about 35%, between about 35% and about 55%, between about 35% and about 50%, between about 35% and about 45%, between about 35% and about 40%, between about 40% and about 55%, between about 40% and about 50%, between about 40% and about 45%, between about 45% and about 55%, between about 45% and about 50%, or between about 50% and about 55%.

pH

During the culturing step, the pH of the cell culture can be maintained at a specific pH value by the addition of a base solution, such as an alkali base solution. The pH of a cell culture can be maintained at a pH of between about 6.5 and about 7.5, between about 6.5 and about 7.4, between about 6.5 and about 7.3, between about 6.5 and about 7.2, between about 6.5 and about 7.1, between about 6.5 and about 7.0, between about 6.5 and about 6.9, between about 6.5 and about 6.8, between about 6.5 and about 6.7, between about 6.6 and about 7.5, between about 6.6 and about 7.4, between about 6.6 and about 7.3, between about 6.6 and about 7.2, between about 6.6 and about 7.1, between about 6.6 and about 7.0, between about 6.6 and about 6.9, between about 6.6 and about 6.8, between about 6.7 and about 7.5, between about 6.7 and about 7.4, between about 6.7 and about 7.3, between about 6.7 and about 7.2, between about 6.7 and about 7.1, between about 6.7 and about 7.0, between about 6.7 and about 6.9, between about 6.8 and about 7.5, between about 6.8 and about 7.4, between about 6.8 and about 7.3, between about 6.8 and about 7.2, between about 6.8 and about 7.1, between about 6.8 and about 7.0, between about 6.9 and about 7.5, between about 6.9 and about 7.4, between about 6.9 and about 7.3, between about 6.9 and about 7.2, between about 6.9 and about 7.1, between about 7.0 and about 7.5, between about 7.0 and about 7.4, between about 7.0 and about 7.3, between about 7.0 and about 7.2, between about 7.1 and about 7.5, between about 7.1 and about 7.4, between about 7.1 and about 7.3, between about 7.2 and about 7.5, between about 7.2 and about 7.4, or between about 7.3 and about 7.5. In some examples, the pH of the culture is maintained at pH 7.00 with a dead zone off 0.05 (a pH of between about 6.95 and about 7.05).

### Recombinant Proteins

Non-limiting examples of recombinant proteins in any of the methods described herein are immunoglobulins, including light and heavy chain immunoglobulins, antibodies, including humanized antibodies that bind specifically to human complement protein C5, such as eculizumab, or antibody fragments, enzymes, such as α-galactosidase, Myozyme, Cerezyme, non-antibody proteins, such as human erythropoietin, tumor necrosis factor (TNF), interferon alpha or beta, or immunogenic or antigenic proteins or protein fragments for use in a vaccine. In some embodiments, the recombinant protein is an engineered protein that contains at least one multifunctional recombinant protein scaffold. See, for example, the recombinant antigen-binding proteins described in Gebauer et al., Current Opin. Chem. Biol. 13:245-255, 2009; and U.S. Patent Application Publication No. 2012/0164066. Non-limiting examples of recombinant proteins that are antibodies include: panitumumab, omalizumab, abagovomab, abciximab, actoxumab, adalimumab, adecatumumab, afelimomab, afutuzumab, alacizumab, alacizumab, alemtuzumab, alirocumab, altumomab, amatuximab, anatumomab, apolizumab, atinumab, tocilizumab, basilizimab, bectumomab, belimumab, bevacizumab, biciromab, canakinumab, cetuximab, daclizumab, densumab, eculizumab, edrecolomab, efalizumab, efungumab, ertumaxomab, etaracizumab, golimumab, infliximab, natalizumab, palivizumab, panitumumab, pertuzumab, ranibizumab, rituximab, tocilizumab, and trastuzumab.

The recombinant protein produced by the mammalian cells cultured in any of the methods described herein can be a recombinant eculizumab including a heavy chain including SEQ ID NO: 1 and a light chain including SEQ ID NO: 2. The recombinant protein produced by the mammalian cells cultured in any of the methods described herein can be a recombinant eculizumab including a heavy chain of SEQ ID NO: 1 and a light chain of SEQ ID NO: 2. Nucleic acid that encodes the heavy and light chains of eculizumab are known in the art (see, for example, the nucleic acid sequences in U.S. Pat. No. 6,355,245 and Fc region sequences in An et al., mAbs 1:6, 572-579, 2009).

Additional examples of therapeutic antibodies that can be produced by the methods described herein are known in the art.

### Collecting the Recombinant Protein

Some embodiments of any of the methods described herein further include a step of collecting the recombinant protein produced in the culturing step. In some examples, the collecting includes lysing the mammalian cells. In some examples, the recombinant protein is collected from the culture medium (e.g., one or both of the first liquid culture medium and feed culture medium for methods including a fed batch culturing step, or one or both of the first liquid culture medium and the second liquid culture medium for methods including a perfusion culturing step). Methods for lysing a mammalian cells are well known in the art. Methods for collecting a recombinant protein from culture media are well-known in the art, and include, e.g., affinity chromatography and/or filtration.

### Purifying the Collected Recombinant Protein

Some embodiments of any of the methods described herein further include a step of purifying the collected recombinant protein. As is known in the art, a collected recombinant protein can be purified using methods known in the art. For example, one or more steps of filtration and chromatography (e.g., affinity chromatography (e.g., using a protein A resin), anionic exchange chromatography, cation exchange chromatography, molecular sieve chromatography, and hydrophobic interaction chromatography) can be formed to purify the collected recombinant protein. Additional methods for purifying a collected recombinant protein (e.g., a shifted recombinant protein product) are well known in the art.

The purified recombinant protein can be substantially free (such as at least or about 90% free, or about 95%, 96%, 97%, 98%, or at least or about 99% free) of contaminating proteins from a liquid culture medium and/or contaminating DNA, proteins, lipids, and carbohydrates from the lysate of a mammalian cell.

### Shifting the Isoelectric Profile of the Collected or Purified Recombinant Protein

In some examples, the isoelectric profile of the collected or purified recombinant protein may be shifted (shifted to a more acidic isoelectric profile) using the methods described in U.S. Provisional Patent Application No. 62/064,397, filed Oct. 15, 2014, which is incorporated by reference herein.

### Formulating the Recombinant Protein

Some embodiments of any of the methods described herein further include formulating the collected recombinant protein, the purified recombinant protein, or the shifted recombinant protein into a pharmaceutical composition. Formulating the collected recombinant protein, the purified recombinant protein, or the shifted recombinant protein into a pharmaceutical composition can include the step of mixing or adding the purified recombinant protein, the collected recombinant protein, or the shifted recombinant protein to a pharmaceutically acceptable excipient to generate the pharmaceutical composition. Examples of pharmaceutically acceptable excipients (e.g., non-naturally occurring pharmaceutically acceptable excipients) are well known in the art.

### EXAMPLES

The invention is further described in the following examples, which do not limit the scope of the invention described in the claims.

### Example 1

### Preparation of a Non-Enhanced BSA

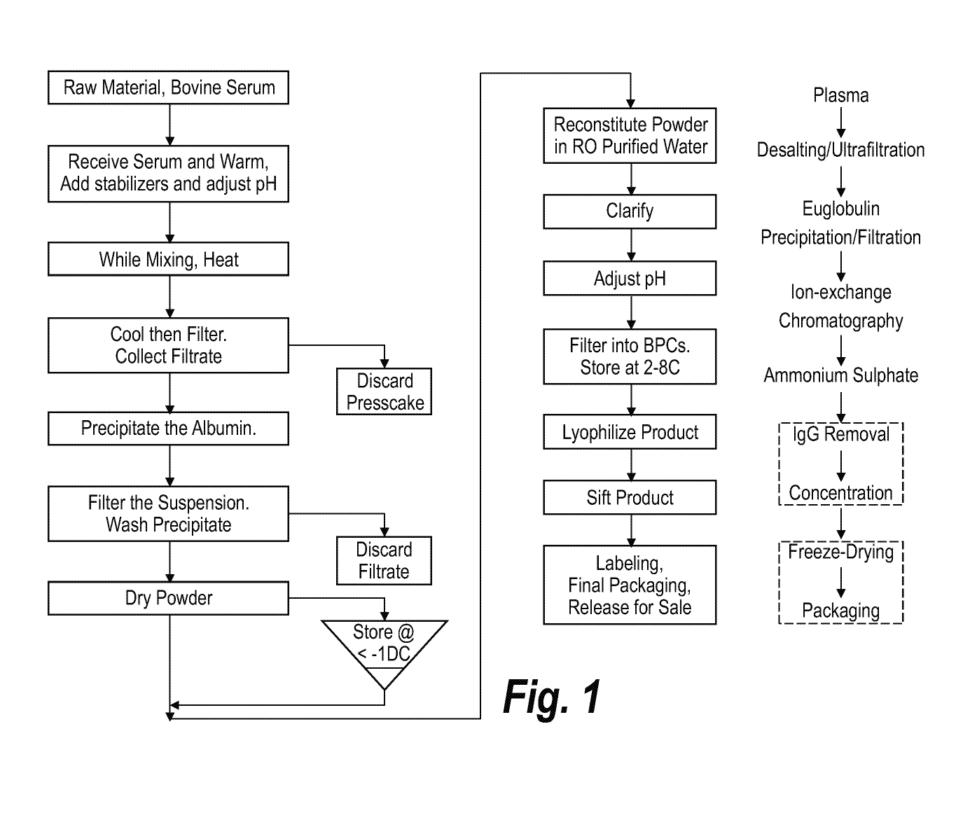

Non-enhanced BSA can be purchased through several commercial vendors. For example, EMD Millipore Probumin®, is an example of a non-enhanced BSA that is commercially available. Probumin® is generated using the method depicted in the left and middle flow charts shown in FIG. 1. The left flow chart in FIG. 1 shows the upstream steps used to generate Probumin®. The upstream steps used to generate Probumin® are (sequentially): warming the plasma from a bovine, adding stabilizers and adjusting the pH after warming, heating the plasma while mixing, cooling and filtering the plasma, collecting the filtrate after filtration, precipitating albumin from the filtrate, filtering the filtrate to remove the precipitated albumin, washing the precipitated albumin, drying the precipitated albumin, reconstituting the precipitated albumin in reverse osmosis purified water to provide a solution containing serum albumin, clarifying the solution, adjusting the pH of the solution, filtering the solution, lyophilizing the filtered solution and sifting the resulting lyophilized material containing serum albumin. The methods used to perform each step in generating Probumin® are generally well-known in the art.

### Example 2

### Preparation of Processed BSA

An example of a processed BSA is MP Biomedicals NZ BSA. MP Biomedicals NZ BSA is generated using the method depicted in the right flow chart in FIG. 1. The steps used to generate MP Biomedicals NZ BSA are (sequentially): desalting a plasma from a bovine, filtering the plasma using ultrafiltration, precipitating euglobulin out of the plasma, filtering the plasma to remove the precipitated euglobulin, performing ion exchange chromatography on the plasma to provide an eluate comprising serum albumin and immunoglobulins, precipitating immunoglobulins out of the eluate using ammonium sulfate precipitation, removing the precipitated immunoglobulins from the eluate, concentrating and freeze-drying the eluate to produce a lyophilized material comprising serum albumin, and optionally, reconstituting the lyophilized material into a solution. The methods used to perform each step in generating MP Biomedicals NZ BSA are generally well-known in the art.

### Example 3

### Effect of Processed and Non-Enhanced BSA on NS0 Cell Growth, Cell Viability, and Productivity in a Fed-Batch Culture

A set of experiments were performed to determine the effect of a processed BSA (MP Biomedicals NZ BSA) and a non-enhanced BSA (EMD Millipore Probumin®) on NS0 cell growth, cell viability, and productivity in a 10,000 fed-batch production cell culture. The NS0 cells in each cell culture contain a nucleic acid encoding recombinant eculizumab. The same culturing parameters were used for each culture. One set of cultures were cultured using liquid culture media containing the processed BSA and another set of cultures were cultured using liquid culture media containing non-enhanced BSA. The viable cell density, the percentage cell viability, and the titer of secreted recombinant eculizumab present in the liquid culture medium were measured over the duration of each cell culture using methods known in the art.

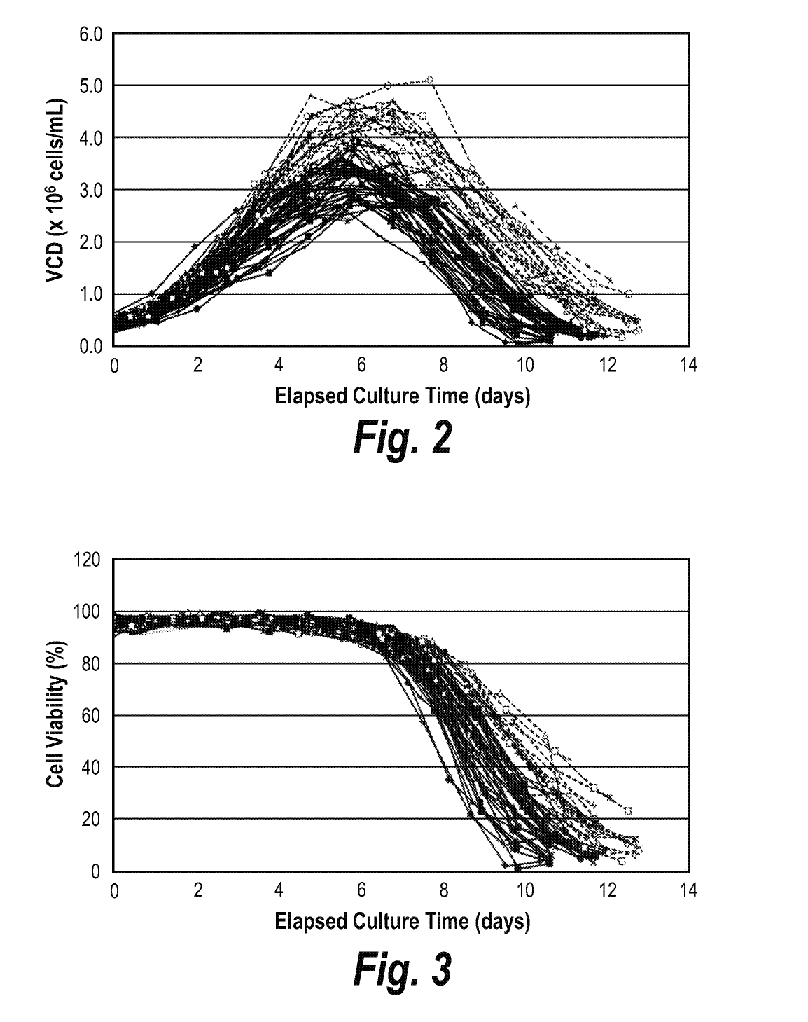

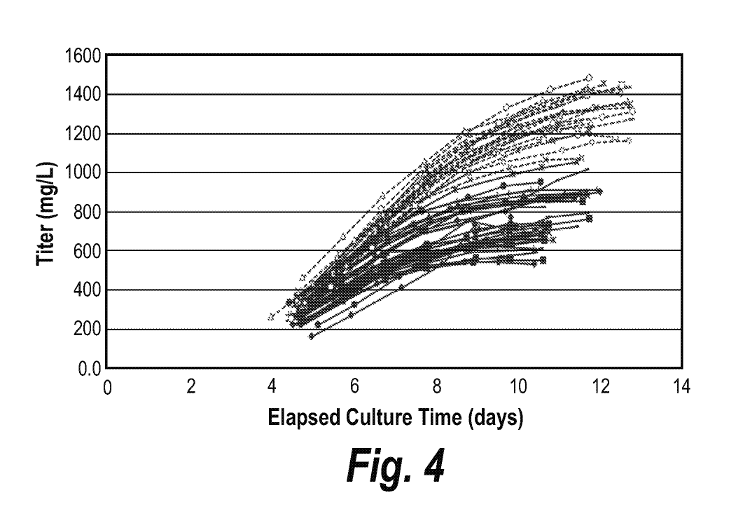

The data in FIG. 2 show that the cells cultured using a liquid culture medium containing a processed BSA demonstrated significantly improved viable cell density over the entire culturing period as compared to cells cultured using a liquid culture medium containing a non-enhanced BSA. The data in FIG. 3 show that, at later time points in the culturing period, cells cultured using a liquid culture medium containing a processed BSA have an increased percentage viable cell density as compared to cells cultured using a liquid culture medium containing a non-enhanced BSA. The data also show that cells cultured using a liquid culture medium containing a processed BSA produce a higher titer of eculizumab over days 4-12 of culture, as compared to cells cultured using a liquid culture medium containing a non-enhanced BSA (FIG. 4).

In sum, these data show that NS0 cells cultured in a liquid culture medium containing a processed BSA have substantially increased viable cell density, percentage cell viability, and productivity, as compared to NS0 cells cultured in a liquid culture medium containing a non-enhanced BSA.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Sequence Appendix
PRT
Homo sapiens
Eculizumab Heavy Chain
SEQ ID NO: 1
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYC
ARYFFGSSPNWYFDVWGQGTLVTVSSASTKGPSVFPLAPCSRSTSEST
AALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPS
VFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEA
LHNHYTQKSLSLSLGK
PRT
Homo sapiens
Eculizumab Light Chain
SEQ ID NO: 2
DIQMTQSPSSLSASVGDRVTITCGASENIYGALNWYQQKPGKAPKLLI
YGATNLADGVPSRFSGSGSGTDFTLTISSLQPEDFATYYCQNVLNTPL
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC

1. A method of culturing a mammalian cell, the method comprising:
fed batch culturing a NS0 cell comprising a recombinant eculizumab-encoding nucleic acid in a liquid culture medium comprising 0.1 g/L or more processed Bovine Serum Albumin (BSA) under conditions sufficient to produce the recombinant eculizumab, wherein the processed BSA is present in and/or added to the liquid culture medium prior to and/or during the culturing step.

2. The method of claim 1, wherein the processed BSA is produced by a method comprising the steps of:
providing plasma from a bovine;
desalting the plasma;
filtering the plasma using ultrafiltration;
precipitating euglobulin out of the plasma;
filtering the plasma to remove the precipitated euglobulin;
performing ion exchange chromatography on the plasma to provide an eluate comprising serum albumin and immunoglobulins;
precipitating immunoglobulins out of the eluate using ammonium sulfate precipitation;
removing the precipitated immunoglobulins from the eluate;
concentrating and freeze-drying the eluate to produce a lyophilized material comprising serum albumin; and optionally
reconstituting the lyophilized material into a solution.

3. The method of claim 1, further comprising producing the processed BSA performing the steps of:
providing plasma from a bovine;
desalting the plasma;
filtering the plasma using ultrafiltration;
precipitating euglobulin out of the plasma;
filtering the plasma to remove the precipitated euglobulin;
performing ion exchange chromatography on the plasma to provide an eluate comprising serum albumin and immunoglobulins;
precipitating immunoglobulins out of the eluate using ammonium sulfate precipitation;
removing the precipitated immunoglobulins from the eluate;
concentrating and freeze-drying the eluate to produce a lyophilized material comprising serum albumin; and
optionally, reconstituting the lyophilized material into a solution.

4. The method of claim 1, wherein the processed BSA is produced by a method that does not include heating a solution comprising serum albumin, adding a stabilizer to a solution comprising serum albumin, or precipitating impurities out of a solution that comprises a reconstituted lyophilized serum albumin.

5. The method of claim 1, wherein the processed BSA is a processed New Zealand BSA.

6. The method of claim 5, wherein the processed NZ BSA is MP Biomedical NZ Limited NZ BSA.

7. The method of claim 1, wherein the liquid culture medium comprises at least 0.5 g/L processed BSA.

8. The method of claim 1, wherein the method comprises adding processed BSA to the liquid culture medium prior to and/or during the culturing step to provide a concentration of 0.1 g/L or more processed BSA in the culture medium.

9. The method of claim 1, wherein the processed BSA is present in the liquid culture medium prior to the culturing step.

10. The method of claim 1, further comprising collecting the recombinant eculizumab produced in the culturing step.

11. The method of claim 10, wherein collecting comprises lysing the NS0 cells.

12. The method of claim 10, wherein recombinant eculizumab is collected from the medium.

13. The method of claim 10, further comprising formulating the collected recombinant eculizumab into a pharmaceutical composition.

14. The method of claim 1, wherein eculizumab comprises a heavy chain comprising SEQ ID NO: 1 and a light chain comprising SEQ ID NO: 2.

15. The method of claim 14, wherein eculizumab comprises a heavy chain consisting of SEQ ID NO: 1 and a light chain consisting of SEQ ID NO: 2.

16. The method of claim 2, wherein eculizumab comprises a heavy chain comprising SEQ ID NO: 1 and a light chain comprising SEQ ID NO: 2.

17. The method of claim 16, wherein eculizumab comprises a heavy chain consisting of SEQ ID NO: 1 and a light chain consisting of SEQ ID NO: 2.

18. The method of claim 3, wherein eculizumab comprises a heavy chain comprising SEQ ID NO: 1 and a light chain comprising SEQ ID NO: 2.

19. The method of claim 18, wherein eculizumab comprises a heavy chain consisting of SEQ ID NO: 1 and a light chain consisting of SEQ ID NO: 2.


SEQUENCE LISTING (2 sequences)

SEQ ID NO: 1 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence [sequence omitted]